(19) United States
(12) Patent Application Publication
(10) Pub. No.: US 2024/0343671 A1
(43) Pub. Date: Oct. 17, 2024

(54) METHOD FOR LACTIDE AND LACTIC ACID RECOVERY AT POLYLACTIDE (PLA) PRODUCTION STEPS

(71) Applicant: FUTERRO S.A., Escanaffles (BE)

(72) Inventors: Philippe COSZACH, Escanaffles (BE); Martin VAN GANSBERGHE, Escanaffles (BE)

(21) Appl. No.: 18/293,745

(22) PCT Filed: Aug. 1, 2022
(86) PCT No.: PCT/EP2022/071579

§ 371 Date: Jan. 30, 2024

(30) Foreign Application Priority Data
Aug. 2, 2021 (BE) 20210054

(51) Int. Cl. C07C 51/42; B01D 1/22; B01D 3/14; B01D 5/00; B01D 9/00; C07D 319/12; C08G 63/08; C08G 63/78

(52) U.S. Cl. CPC C07C 51/42; B01D 1/22; B01D 3/148; B01D 5/003; B01D 9/0009; C07D 319/12; C08G 63/08; C08G 63/78; B01D 2009/0086

(57) ABSTRACT

An improved method is described for producing polylactide (PLA) whereby various gas streams containing lactide, lactic acid and water are recovered, are sent onto an assembly of packed columns for condensation, are brought into contact with a fluid at least comprising water, lactic acid and dissolved lactide; the fluid leaving the assembly of packed columns is recovered and is or is not filtered, the aqueous solution obtained via at least one exchanger is heated and sent into a feed tank under agitation which is continuously fed with a lactic acid solution, and hydrolysis of the lactide contained in the aqueous solution is performed in this feed tank.

### TECHNICAL FIELD

The present disclosure relates to a method for producing polylactide (PLA) and in particular to an improved method which allows maximum recovery of useful materials for loss-free recycling and therefore an improvement in the overall yield of the PLA production method starting from lactic acid. More specifically, the present disclosure relates to a method allowing a solution to be found to the problem of recycling lactide-containing gas streams.

### BACKGROUND

It is known that among usual methods for producing PLA, there is a first type entailing direct polycondensation of lactic acid, as described in JP 733861; but this type of method is limited through the use of a solvent and hence difficulties in removing water from the reaction mixture Another type of usual method for producing PLA starting from lactic acid comprises several steps to oligomerize the lactic acid to oligomers, to cyclize oligomers to lactide, to purify the crude lactide and to polymerize the purified lactide to PLA, before demonomerizing and devolatilizing the PLA obtained to extract non-converted lactide therefrom before the final step which comprises granulation, crystallization and drying of the PLA granules.

Throughout these steps of methods for preparing PLA, a whole series of secondary streams are generated leading to substantial losses of material such as at the time of purging and withdrawal of vapors which are essentially at reduced pressure of about 0.1 mbar to about 400 mbar and more specifically of about 1 mbar to about 200 mbar, and which notably contain water, lactic acid, lactide and other small oligomers of lactic acid or esters of lactic acid having different compositions.

It has also been ascertained that with these usual methods, taking as reference a theoretical method without any recycling, the overall yield is of about 50 molar %.

It has already been envisaged to improve this yield by controlling the so-called easy recycling of some streams such as those of oligomerization condensates or lactide purification which alone allow the overall yield to be increased to about 75%.

However, these yields remain insufficient for the conducting of an industrial process.

The applicant, to improve the overall yield of the process, has already proposed acting at the purification stage of by-products from the lactide purification steps via distillation to extract therefrom a mixture of purified lactide, the stream thus generated being recycled back to the main stream to reduce losses and modulate the -D-enantiomer content of the produced PLA as described in WO2014/180836. The Applicant has also proposed recovering a maximum number of lactate units from secondary streams via an esterification/trans-esterification/distillation process and hydrolysis of the ester to lactic acid as described in WO2015/086613.

In addition, and as set forth above, a non-negligible part of purging and draw-off takes place in vapor form, and it is also well known in the prior art that these vapors can be recovered and reconverted first to lactide and then to lactic acid via simple hydrolysis.

It is therefore possible to recover the vapors of products at each of the steps of the method, and first to subject these vapors to condensation which is most often performed indirectly by passing the gas stream over cold surfaces.

U.S. Pat. No. 5,266,706 B2 is also known which describes a method for recovering lactide from a gas stream containing inter alia lactic acid and water, by scrubbing this gas stream with a water-immiscible solvent such as nonpolar, cycloaliphatic or halogenated hydrocarbons. In this process, the temperature is adjusted during scrubbing in a manner to recover water, lactide and lactic acid. However, a final step must necessarily be dedicated to separating the solvent.

To overcome the problems generated by the presence of solvent, mention can be made of U.S. Pat. No. 8,430,948 B2 which shows another manner of treating the gas stream containing lactide, lactic acid and water. With this process, the vapor stream is transferred via a line to feed a condensation column where it is recovered in the receptacle positioned at the bottom of this column. A liquid phase is formed therein containing dissolved lactide, lactic acid, as well as condensation and scrub water.

The lactic acid present at the bottom of this condensation column is pumped via an exchanger to be sent towards the distributor of the condensation column.

The lactic acid falls onto a packed bed and mixes with the process gas stream.

The method described in U.S. Pat. No. 8,430,948 B2 is adjusted so that the lactide is dissolved at the bottom of the column, without the content of lactide dissolved in the liquid medium (water+lactic acid and lactide) exceeding 5% by weight.

The non-condensable products are evacuated at the head of the column via the line towards the vacuum unit.

In addition, the notion of condensation of the lactide contained in the vapor phase is also described in FR 2726559 which describes a method for treating this vapor phase stream containing inter alia lactide. It comprises entraining by water vapor, condensing the lactide, lactic acid and water vapors, and the forming of an aqueous solution in which lactide is dissolved and precipitates.

In this respect, U.S. Pat. No. 9,700,840 B2 can also be cited, which relates more particularly to the treatment of the aqueous solution after condensation of the vapor phase, using a process whereby this aqueous solution is successively subjected to heating, reaction and cooling under the strict conditions of preventing any external contribution of lactic acid into the system and any precipitation of lactide within the circuit.

It can be seen that while solutions exist for ensuring the recycling of the lactide to lactic acid at the different steps of the PLA preparation method, these solutions comprise operating conditions which can curb the recovery of the lactide and lactic acid for reuse thereof.

Particular mention is made of the adjustment of lactide dissolution, or the exclusion of precipitation of the lactide in the aqueous solution, which result in limiting the overall yield of PLA production and amount to a major drawback for an industrial process.

There is therefore a need for the implementation of a method allowing restriction-free recycling of the secondary lactide and lactic acid streams derived from the different steps of the PLA preparation method, whilst allowing a very high overall yield without impacting method flexibility.

### SUMMARY

The Applicant has found a method with which it is possible to reduce the above-mentioned drawbacks, this method comprising the steps of:
providing, together or separately, various gas streams containing lactide, lactic acid and water;
sending this gas stream onto an assembly of packed columns comprising at least one column, in which it will be condensed;
bringing this gas stream, in this assembly of columns, into contact with a wetting fluid at a temperature comprised between 1° C. and 30° C., this fluid comprising water, lactic acid and dissolved lactide;
recovering the fluid leaving the assembly of packed columns at a temperature comprised between 15° C. and 70° C. and preferably between 20° C. and 60° C., this fluid inter alia containing lactide in solid form in an amount comprised between 0.1% and 20% by weight relative to the total weight of lactates contained in solution, preferably between 1% and 10% by weight, and more preferably between 2% and 5% by weight relative to the total weight of lactates contained in solution;
optionally, filtering and extracting at least partially the precipitated lactide contained in this fluid;
heating this aqueous solution, from which solid lactide may have been partially removed, via at least one exchanger to bring the temperature to a value comprised between 50° C. and 95° C. and preferably between 60° C. and 80° C.;
sending this aqueous solution to a feed tank under agitation and continuously fed with a stream of lactic acid, this feed tank being positioned upstream of the present treatment circuit and being held at a temperature of between 50° C. and 95° C., and preferably between 60° C. and 80° C.;
in this feed tank, performing hydrolysis of the lactide contained in the aqueous solution for conversion thereof to lactic acid.

It is understood that according to the method of the present disclosure, it is lactic acid and/or lactic acid oligomers and more specifically dimers of lactic acid that are recovered, but the lactic acid may also be found in the form of an ester of lactic acid such as methyl, ethyl, butyl lactate or others.

### DETAILED DESCRIPTION

The method of the present disclosure is also described by means of the following figures but is not to be interpreted as being limited thereby:

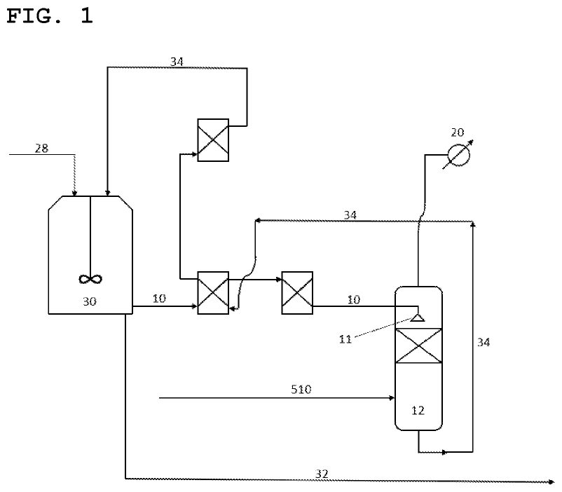

FIG. 1 schematically describes the method of treating the various gas streams derived for example from oligomerization, cyclization, purification of the lactide and devolatilization of the PLA, which are either collected in a single stream or treated separately to be subjected to a series of operations for recovery of lactic acid or light oligomer of lactic acid, this treatment comprising at least one packed column 12 surmounted by a vacuum unit 20.

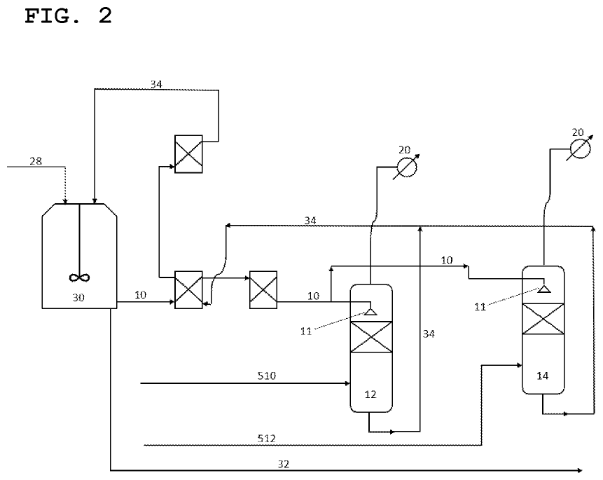

FIG. 2 schematically describes one particular embodiment of the same method but in which the packed columns 12 and 14 are connected in parallel.

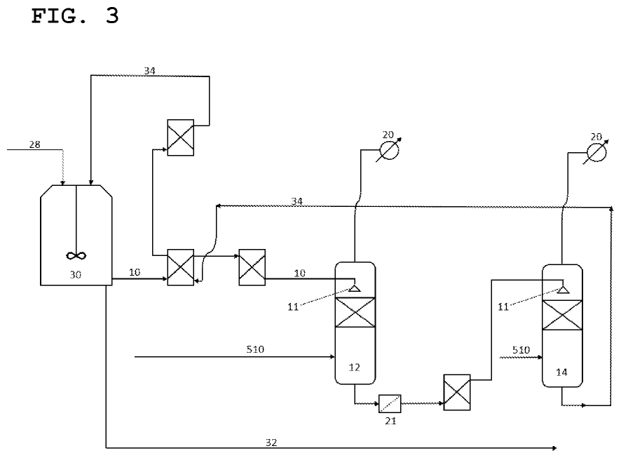

FIG. 3 schematically describes another particular embodiment of the same method but in which the packed columns 12 and 14 are connected in series.

In FIG. 1, a method is described wherein the various gas streams containing lactide, lactic acid and water are treated by passing them together or separately through the line 510 to reach the feed inlet of the packed column 12; the feed inlet to this column 12 is positioned below the distributor 11; the packed column 12 is also fed with the acid aqueous solution derived from the feed tank 30 via line 10 after having passed through a series of exchangers to lower the temperature thereof to a value comprised between 1° C. and 30° C., preferably between 1° C. and 20° C., more preferably between 5° C. and 15° C., before being distributed into the column 12 via the distributor 11. At the foot of the column 12, an aqueous solution is drawn off containing lactic acid, water, dissolved lactide and precipitated lactide, this solution having a temperature comprised between 15° C. and 70° C. and it is recycled, after passing through at least one exchanger (two or more exchangers can be envisaged to optimize energy recovery) to bring the temperature to a value comprised between 50° C. and 95° C., back to the feed tank 30 via line 34. The feed tank 30, in addition to the recycled aqueous solution, is continuously fed with a lactic acid solution via line 28; hydrolysis of the lactide to lactic acid or light oligomer of lactic acid takes place in this feed tank 30. In addition, a portion of the acid aqueous solution is withdrawn from this feed tank 30 and sent to the evaporation step via line 32 to recover concentrated lactic acid, while another portion feeds the packed column 12 via line 10.

The aqueous solution circulating between the packed column 12 and the feed tank 30 contains precipitated lactide in an amount comprised between 0.1% and 20% by weight relative to the total weight of lactates contained in solution.

With this method, it is also possible when drawing off at the bottom of the column 12, to filter the solution in batch or continuous mode for partial recovery of the precipitated lactide (not illustrated in FIG. 1).

In the particular embodiment of the method described in FIG. 2, a packed column 14 has simply been added to the first column 12 for treatment of the gas streams, these columns being arranged in parallel. They are fed with the gas streams containing lactide, lactic acid and water via lines 510 and 512, and with the acid aqueous solution derived from the feed tank 30 via line 10.

An aqueous solution is drawn off containing lactic acid, water, dissolved lactide and precipitated lactide at the bottom of columns 12 and 14, and it is sent towards at least one exchanger and preferably two exchangers to bring the temperature to a value comprised between 50° C. and 95° C., preferably between 55° C. and 85° C., before being recycled back to the feed tank 30 via line 34, this feed tank in addition to this acid aqueous solution being continuously fed with a lactic acid solution via line 28. The lactides contained in this feed vessel 30 are hydrolyzed to lactic acid and/or lactic acid oligomer. Solid lactide is found between the bottom of columns 12 and 14 and the feed tank 30 in an amount of 0.1% to 20% by weight relative to the total weight of lactates contained in solution and preferably from 1% to 10% by weight relative to the total weight of lactates contained in solution, in addition to the dissolved lactide. Also, a portion of the acid aqueous solution is drawn off from this feed tank 30 and sent to the evaporation step via line 32 to recover concentrated lactic acid, whilst another portion via line 10 feeds the packed columns 12 and 14.

In the particular embodiment described in FIG. 3, the gas stream is treated by means of two packed columns that are here connected in series. In this configuration of the device, a filtering (via filter 21) and partial extraction operation of the precipitated lactide drawn-off at the foot of the packed column 12 proves to be necessary, in batch or continuous mode, and even essential. In batch mode, it is also possible to place two filters in parallel to allow continuous operation. However, the aqueous solution circulating between the packed column 14 and the feed tank 30 necessarily contains precipitated lactide in an amount of between 0.1% and 20% by weight relative to the total weight of lactates contained in solution.

The aqueous solution containing lactic acid, water, dissolved lactide and precipitated lactide is drawn off from the second column 14 and sent towards at least one exchanger to bring the temperature thereof to a value comprised between 50° C. and 95° C., preferably between 60° C. and 75° C. before being returned to the feed tank 30 which, in addition to this acid aqueous solution, is continuously fed via line 28 with a lactic acid solution. The lactides contained in this feed tank 30 are hydrolyzed to lactic acid and/or lactic acid oligomer; also, a portion of the acid aqueous solution is drawn off from this feed tank 30 and sent to the evaporation step via line 32 to recover concentrated lactic acid, whilst another portion feeds the packed column 12 via line 10.

It is thus shown that with the method of the present disclosure it is possible, without blocking the circuit, to recover the most possible secondary streams whilst avoiding the drastic conditions for recovery of lactide as taught in the patents in the prior art.

The method of the present disclosure therefore allows more secondary streams to be recovered so that they can be almost entirely recycled back into the PLA preparation method or into other operations, without any loss.

In one embodiment of the method of the present disclosure, the lactic acid recovered in the feed tank 30 can subsequently be treated first by pre-concentration performed on a multiple effect evaporator followed by thin film concentration and finally distillation according to the method described in BE 1011197, to obtain a lactic acid of polymer grade quality.

The recovered polymer grade lactic acid can then be recycled back into the PLA production method described in WO2015/086613 (for example at the oligomerization stage) thereby ensuring high yields by reducing losses.

The method of the present disclosure is also described and illustrated in the following nonlimiting examples.

### Example 1

PLA was prepared following the method described in WO2015/086613 by the applicant. This method already makes provision for some recycling but not in accordance with the method of the present disclosure.

The secondary streams were collected in vapor phase as described in FIG. 3, these streams containing lactide, lactic acid and water, and were grouped together in line 510 and sent towards the packed columns 12 and 14 connected in series. These streams were then condensed and brought into contact with an acid aqueous solution derived from feed tank 30 that was conveyed via line 10 to the distributor 11 of the packed column 12. The aqueous solution collected at the bottom of column 12 was filtered for partial removal of solid lactide, 55% of the solid lactide was therefore removed. The aqueous solution, after being cooled in an exchanger, was sent to the packed column 14 to be distributed by the distributor 11, and was also brought into contact with another part of the gas stream 510 (containing water, lactic acid and lactide).

The aqueous solution containing lactic acid, dissolved lactide and precipitated lactide in addition to water was drawn off the second column 14 (heated to 20° C.) and sent towards two exchangers, the first being intended for the recovery of energy, to bring the temperature thereof to 70° C. before it was recycled back to the feed tank 30 which, in addition to this acid aqueous solution was continuously fed via line 28 with fresh lactic acid solution. The lactides contained in this feed tank 30 were hydrolyzed to lactic acid. In the part of the circuit lying between the foot of column 14 and the feed tank 30, solid lactide was found in an amount of 2.4% by weight relative to the total weight of lactates present in solution, in addition to the dissolved lactide and added fresh lactic acid.

The lactic acid recovered in the feed tank 30 was afterwards treated by undergoing concentration on a multiple effect evaporator followed by passing through a thin film evaporator and finally distillation conforming to the method described in BE1011197, to obtain polymer grade lactic acid. The recovered polymer grade lactic acid was then recycled without any restriction at the oligomerization step of the PLA production method described in WO2015/086613. The Lactide/PLA yield was higher although close to that of this patent, but more especially allowed reuse of all recycling thereby limiting losses overall.

### Example 2

As in Example 1, PLA was prepared following the method described in patent WO2015/086613 by the Applicant. The secondary streams were collected in vapour phase as described in FIG. 2, these streams containing lactide, lactic acid and water, and were grouped together in lines 510 and 512 and sent to the packed columns 12 and 14 connected in parallel. These streams were condensed and brought into contact with an acid aqueous solution derived from feed tank 30 and conveyed via line 10 to the distributor 11 of the packed columns 12 and 14.

The aqueous solution containing lactic acid, dissolved lactide and some precipitated lactide (in an amount of respectively 1.4% and 1.6% by weight relative to the total weight of the lactates contained in solution) in addition to water, was drawn off from the bottom of columns 12 and 14 and sent to two exchangers, the first being intended for energy recovery, to bring the temperature thereof to 70° C. before it was recycled back to the feed tank 30 via line 34 which, in addition to this acid aqueous solution was continuously fed via line 28 with fresh lactic acid solution. The lactides contained in this feed tank 30 were hydrolyzed to lactic acid and/or lactic acid oligomer.

The lactic acid recovered in the feed tank 30 was then treated by first undergoing concentration on a multiple effect evaporator followed by passing through a thin film evaporator and finally distillation, conforming to the method described in BE1011197 to obtain lactic acid of polymer grade quality.

1. A Polylactide (PLA) preparation method with improved overall yield, of the method comprising:
providing together or separately, various a plurality of gas streams containing lactide, lactic acid, and water;
sending each of the plurality of gas streams onto an assembly of packed columns comprising at least one column, in which the gas stream will be condensed;
bringing the gas stream, in the assembly of packed columns, in contact with a wetting fluid at a temperature of 1° C. to 30° C., this fluid comprising water, lactic acid and dissolved lactide;
recovering the fluid leaving the assembly of packed columns at a temperature of 15° C. to 70° C., this fluid inter alia containing lactide in solid form of 0.1% to 20% by weight relative to a total weight of lactates contained in solution;
optionally, filtering and extracting at least partially the precipitated lactide contained in this fluid;
heating this aqueous solution, from which solid lactide may have been partially removed, via at least one exchanger, to bring the temperature to a value comprised between 50° C. and 95° C.;
sending this acid aqueous solution into a feed tank under agitation and continuously fed with a stream of lactic acid, this feed tank being positioned upstream of the present treatment circuit and being held at a temperature of 50° C. to 95° C.; and
in this feed tank, converting the lactide contained in the aqueous solution to lactic acid and/or lactic acid oligomer by performing hydrolysis.

2. The method according to claim 1, wherein the packed columns are connected in parallel.

3. The method according to claim 1, wherein the packed columns are connected in series.

4. The method according to claim 3, wherein precipitated lactide is filtered and at least partially extracted at a bottom of a first packed column.

5. The method according to claim 1, wherein the wetting fluid is at a temperature of 5° C. to 12° C.

6. The method according to claim 1, wherein the fluid leaving the assembly of packed columns is at a temperature of 20° C. to 60° C.

7. The method according to claim 1, further comprising:
filtering and extracting at least partially a precipitated lactide contained in the aqueous solution; and
wherein the aqueous solution, from which solid lactide has been partially removed, is heated to a temperature of 55° C. to 85° C.

8. The method according to claim 1, wherein the acid aqueous solution circulating between the packed columns and the feed tank contains lactide in precipitated form.

9. The method according to claim 8, wherein the aqueous solution contains lactide in precipitated form in an amount of 1 to 10% by weight relative to the weight of lactates contained in the solution.

10. The method according to claim 1, wherein lactic acid recovered after hydrolysis in the feed tank is subjected to concentration via at least one thin film evaporator, and to distillation to obtain lactic acid of polymer grade quality.

11. The method of claim 1, further comprising filtering and extracting at least partially a precipitated lactide contained in the aqueous solution.
